(19) United States
(12) Patent Application Publication
(10) Pub. No.: US 2024/0197802 A1
(43) Pub. Date: Jun. 20, 2024

(54) METHOD FOR HEAVY METAL DETOXIFICATION BY CRASSOCEPHALUM RABENS EXTRACT

(71) Applicant: Greenyn Biotechnology Co., Ltd, Taichung City (TW)

(72) Inventors: Pang-Kuei HSU, Taichung City (TW); Yu-Cheng LIN, Taichung City (TW); Chia-Feng WU, Taichung City (TW)

(21) Appl. No.: 18/534,140

(22) Filed: Dec. 8, 2023

Related U.S. Application Data
(60) Provisional application No. 63/432,897, filed on Dec. 15, 2022.

(51) Int. Cl. A61K 36/28; A61K 31/7032; A61P 39/00

(52) U.S. Cl. CPC A61K 36/28; A61K 31/7032; A61P 39/00; A61K 2236/331; A61K 2236/333

(57) ABSTRACT

A use of a Crassocephalum rabens extract in detoxification is disclosed. The Crassocephalum rabens extract has the activity to remove such substances in an organism or the cells thereof as a heavy metal, so the administration of an effective amount of the Crassocephalum rabens extract or of a composition containing the Crassocephalum rabens extract to an individual can effectively reduce the heavy metal content in the individual's body and help restore the individual's liver functions.

### BACKGROUND OF THE INVENTION

### 1. Technical Field

The present invention relates to a second use of a plant extract. More particularly, the invention relates to a method for heavy metal detoxification by Crassocephalum rabens extract.

### 2. Description of Related Art

Crassocephalum rabens, also known as Zhaohe Cao, is an edible plant in the family Asteraceae. According to the pharmacopeia of traditional Chinese medicine, Crassocephalum rabens not only can fortify the spleen, relieve swelling, clear away heat, detoxify, facilitate the flow of qi, and induce diuresis, but also can be used to treat hypertension, common cold, dyspepsia, headache, mastitis, and so on. Once pounded and crushed, the stems and leaves of Crassocephalum rabens can serve as a dressing material for treating a mosquito bite or a wound in the skin.

To separate the effective ingredients of herbs in traditional Chinese medicine and incorporate those ingredients into different products, it is common practice to carry out the separation by the solvent extraction method, which takes advantage of the difference in solubility of an effective ingredient in different solvents. However, while the conventional solvent extraction method can separate the effective ingredients of herbs, the extracts obtained still have such problems as insufficient purity and residues of the organic extraction solvents used and therefore either fail to exhibit their bioactivity or have biotoxicity.

### BRIEF SUMMARY OF THE INVENTION

The primary objective of the present invention is to provide a method for heavy metal detoxification by Crassocephalum rabens extract. The Crassocephalum rabens extract disclosed herein has the activity to remove the heavy metal in an individual's body and is therefore effective in treating and/or preventing heavy metal poisoning and the related diseases.

To achieve the foregoing objective, the present invention provides a second use of a Crassocephalum rabens extract in preparing a composition for treating and/or preventing heavy metal poisoning and organ function damage caused thereby, or for lowering the blood lead concentration. That is to say, the administration of an effective amount of the Crassocephalum rabens extract or of a composition containing the Crassocephalum rabens extract to an individual will be able to lower the heavy metal concentration in the individual's body, improve the individual's liver functions, and thereby treat and/or prevent heavy metal poisoning and the related diseases.

In one embodiment of the present invention, the Crassocephalum rabens extract is a mixture rich in 1,2-di-O-linolenoyl-3-O-β-galactopyranosyl-sn-glycerol (dLGG). To be specific, the Crassocephalum rabens extract is obtained by performing an extraction process on Crassocephalum rabens, and the extraction process uses water or a primary alcohol as an extraction solvent.

The primary alcohol may be methanol or ethanol.

In one embodiment of the present invention, the aforesaid heavy metal poisoning is caused by an individual's direct or indirect contact with a heavy metal or with a substance containing a heavy metal, wherein the heavy metal may be, for example, lead, zinc, or cadmium.

In one embodiment of the present invention, the organ function damage caused by the heavy metal poisoning is damage to the liver functions. Damage to the liver functions may induce liver damage or liver-related diseases and result in an increase in the aspartate aminotransferase (AST) level and the alanine aminotransferase (ALT) level in blood.

### BRIEF DESCRIPTION OF THE SEVERAL VIEWS OF THE DRAWINGS

FIG. 1A is an HPLC chromatogram of the Crassocephalum rabens alcohol extract disclosed herein.

FIG. 1B is an HPLC chromatogram of the Crassocephalum rabens water extract disclosed herein.

FIG. 2 is an HPLC chromatogram of a dLGG (1,2-di-O-linolenoyl-3-O-β-galactopyranosyl-sn-glycerol) standard.

FIG. 3 shows the blood lead concentrations of differently treated rats.

FIG. 4 shows the AST and ALT levels in the blood of differently treated rats.

### DETAILED DESCRIPTION OF THE INVENTION

The present invention discloses a method for heavy metal detoxification by Crassocephalum rabens extract. The Crassocephalum rabens extract disclosed herein has the activity to remove such substances in an organism or the cells thereof as a heavy metal, so the administration of an effective amount of the Crassocephalum rabens extract or of a composition containing the Crassocephalum rabens extract to an individual can effectively reduce the heavy metal content in the individual's body and help restore the individual's liver functions.

In one embodiment of the present invention, the Crassocephalum rabens extract is a mixture rich in 1,2-di-O-linolenoyl-3-O-β-galactopyranosyl-sn-glycerol (dLGG) and obtained by performing an extraction process on a raw material of Crassocephalum rabens. The extraction process by which the Crassocephalum rabens extract is obtained may use different extraction solvents such as water, a primary alcohol, and carbon dioxide, and the extraction process may be carried out by a supercritical extraction method, an alcohol extraction method, a water extraction method, or a combination of any two of the aforesaid methods.

The raw material of Crassocephalum rabens is obtained by blanching fresh Crassocephalum rabens.

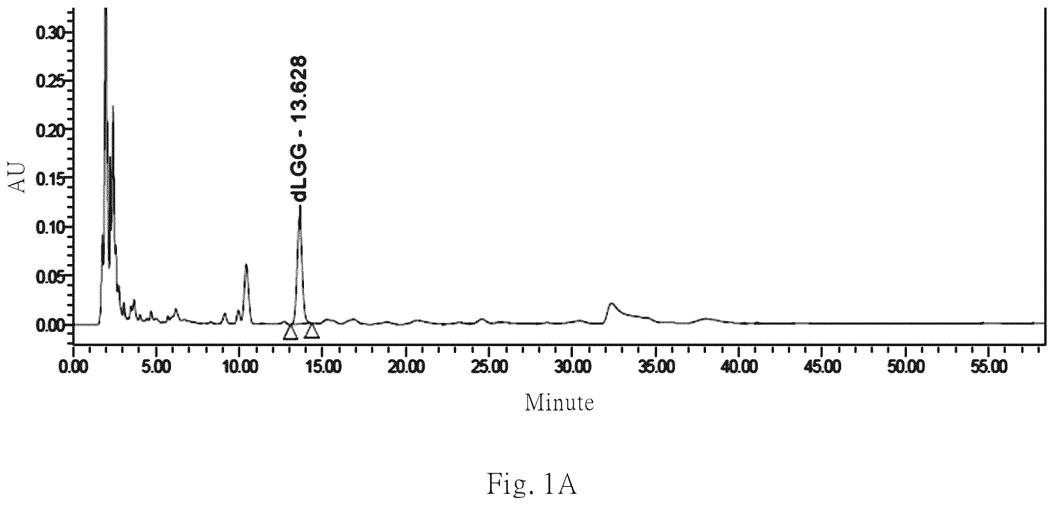

FIG. 1A and FIG. 1B are chromatograms showing the high-performance liquid chromatography (HPLC) analysis results of a Crassocephalum rabens alcohol extract and a Crassocephalum rabens water extract. The alcohol extraction method by which the Crassocephalum rabens alcohol extract was obtained used an alcohol with a concentration higher than or equal to 95% as the extraction solvent, and the water extraction method by which the Crassocephalum rabens water extract was obtained used water as the extraction solvent. The HPLC analysis conditions are as follows: the solvent was 98% methanol; the flow rate, 1.0 mL/min; the elution method, gradient elution; the injected volume, 10 μL; the running time, 60 minutes; the column temperature, 30° C.; and the detection wavelength, 210 nm.

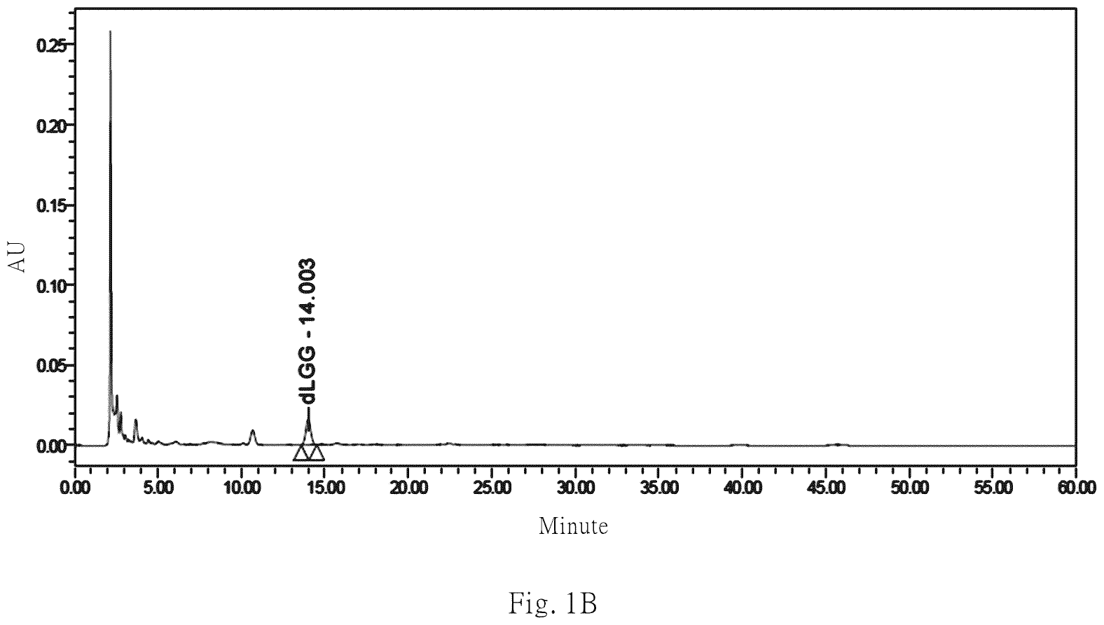

As can be clearly seen in the result in each of FIG. 1A and FIG. 1B, a significant peak occurs in the retention time interval between the 13.5 and 14.5-minute time points. Moreover, a comparison with the HPLC chromatogram of a dLGG standard (see FIG. 2) reveals that the significant peaks in FIG. 1A and FIG. 1B correspond to dLGG. Therefore, it can be inferred that regardless of the method used by the extraction process, the main marker constituent of the resulting Crassocephalum rabens extract is dLGG (1,2-di-O-linolenoyl-3-O-β-galactopyranosyl-sn-glycerol).

In one embodiment of the present invention, the Crassocephalum rabens extract is obtained by sequentially performing a pre-processing process, an extraction process, and a drying concentration process on fresh Crassocephalum rabens, and the Crassocephalum rabens extract obtained can be used to prepare a Crassocephalum rabens composition through a post-processing process that may include compounding.

The pre-processing process is intended to preserve the active ingredients of Crassocephalum rabens and increase the extraction rate of the subsequent extraction process. The pre-processing process includes blanching and crushing. “Blanching” refers to processing fresh Crassocephalum rabens with hot water or water vapor in order to obtain a raw material of Crassocephalum rabens. “Crushing” refers to rendering the raw material of Crassocephalum rabens into 1-μm powder particles, i.e., Crassocephalum rabens powder, with a device such as a cutter or attrition mill.

In the extraction process, an extraction solvent such as water or alcohol is used to separate a Crassocephalum rabens crude extract from the Crassocephalum rabens powder in a low-temperature environment.

The drying concentration process is used to remove moisture from, or lower the water content of, the Crassocephalum rabens crude extract in order to produce the final Crassocephalum rabens extract.

In one embodiment of the present invention, the Crassocephalum rabens composition is intended to be administered to an individual such as a 60-kg human adult, and the effective amount of the Crassocephalum rabens composition is 100-250 mg/day, preferably 200 mg/day. The effective amount may vary with the species, body weight, age, and other parameters of the individual, and any change in the effective amount is encompassed by the appended claims, provided that the change does not exceed a range derivable with an existing technique by a person with common general knowledge in the technical field to which the invention pertains, or that the change falls within a range of allowable error in the same technical field (e.g., the error being within ±10% of the true value). That is to say, an effective amount whose numerical value is not within the range stated above or identical to the preferred value stated above shall still be viewed as falling within the scope of the appended claims.

In one embodiment of the present invention, the individual is a mammal that has been exposed to a heavy metal continuously for a certain period of time, or the cumulative heavy metal content in whose body tissues or organs has exceeded a medically acceptable range.

The heavy metal referred to herein includes lead, arsenic, other metal elements harmful to the human body, and salts of the above.

Unless otherwise defined, all the technical and/or scientific terms used herein have substantially the same connotations or meanings as understood by a person with common general knowledge in the technical field to which the present invention pertains.

The term “extraction process” refers to a process that, by taking advantage of the different solubility of each constituent substance in a mixture, can separate a target substance, or a distinctive portion containing the target substance, from the mixture. The extraction process may use, for example, a water extraction method, an alcohol extraction method, or a supercritical extraction method.

The term “blanching” refers to a process that uses an abrupt change in temperature to preserve the activity of the active ingredients (e.g., the detoxifying ingredients and enzymes) of Crassocephalum rabens, i.e., to keep those ingredients from damage by environmental factors.

The term “composition” refers to a substance that contains the Crassocephalum rabens extract disclosed herein as a main active ingredient, that can be prepared in different dosage forms depending on such factors as the target users and the method of use, and that may be added with an excipient, a vehicle, and/or other ingredients that are pharmaceutically acceptable or are acceptable in the food industry. For example, the composition may be a pharmaceutical, a nutritional supplement, a food item, or a preparation having a therapeutic effect.

The term “effective amount” refers to an amount in which the Crassocephalum rabens extract is administered and that can alleviate one or more symptoms of the disease to be treated, reduce the discomfort of one or more symptoms of the disease, or slow down the progression of the disease. The effective amount may vary with such parameters as the time for which administration continues (or the administration period), the dosage form used for administration, and the individuals to whom the Crassocephalum rabens extract is administered. Any change in the effective amount is encompassed by the appended claims, provided that the change does not exceed a range derivable with an existing technique by a person with common general knowledge in the technical field to which the present invention pertains, or that the change falls within a range of allowable error in the same technical field (e.g., the error being within +10% of the true value). That is to say, an effective amount whose numerical value is not within the range stated herein or identical to the preferred value stated herein shall still be viewed as falling within the scope of the appended claims. For example, when the Crassocephalum rabens extract disclosed herein is administered to a human adult, the daily effective amount of the Crassocephalum rabens extract is 100-250 mg/day, and the administration period is at least 14 days.

The term “treating” refers to reversing, alleviating, or inhibiting the progression or one or more symptoms, of the disease to which the term is applied or preventing the occurrence of the disease or complications thereof. The term “treatment” refers to a therapeutic action of “treating” as defined herein.

The term “administer” refers to providing the Crassocephalum rabens extract disclosed herein or a composition containing the Crassocephalum rabens extract to an individual. The administration may be carried out through external application or orally.

The term “allowable error” means an error between a numerical value and the data or number provided herein that, despite its existence, is scientifically acceptable and does not represent a statistical difference from the data or number disclosed herein or have any impact on the substantial sameness of the corresponding result.

In addition, all the drawings disclosed herein serve only to explain or expound the technical features of the present invention and/or their effects and are not intended to be restrictive of the scope of the appended claims. If a drawing disclosed herein contains a specific numerical value or number, the numerical value or number should be construed as including the corresponding true value and values with an error allowable by a person with common general knowledge in the technical field to which the present invention pertains.

The technical features of the present invention and their effects are detailed below with reference to some examples and the accompanying drawings.

### Example 1: Preparation of the Crassocephalum rabens Extract

Fresh Crassocephalum rabens was sequentially subjected to blanching, crushing, extraction, filtration, centrifugation, hydration, and freeze-drying. Water and 95% alcohol were used separately as the extraction solvent in the extraction process, and the results were a Crassocephalum rabens water extract and a Crassocephalum rabens alcohol extract.

An HPLC analysis was performed on each of the Crassocephalum rabens water extract and the Crassocephalum rabens alcohol extract under the following HPLC conditions: the solvent was 98% methanol; the flow rate, 1.0 mL/min; the elution method, gradient elution; the injected volume, 10 μL; the running time, 60 minutes; the column temperature, 30° C.; and the detection wavelength, 210 nm. The analysis results are presented in FIG. 1A and FIG. 1B, each of which shows a significant peak in the retention time interval between the 13.5 and 14.5-minute time points. A comparison with the HPLC chromatogram of a dLGG standard (see FIG. 2) reveals that the significant peak in the HPLC chromatogram of each of the Crassocephalum rabens water extract and the Crassocephalum rabens alcohol extract corresponds to dLGG (1,2-di-O-linolenoyl-3-O-β-galactopyranosyl-sn-glycerol), and this means that the Crassocephalum rabens water extract and the Crassocephalum rabens alcohol extract were both rich in dLGG.

### Example 2: Detoxification (Lead Removal) Test

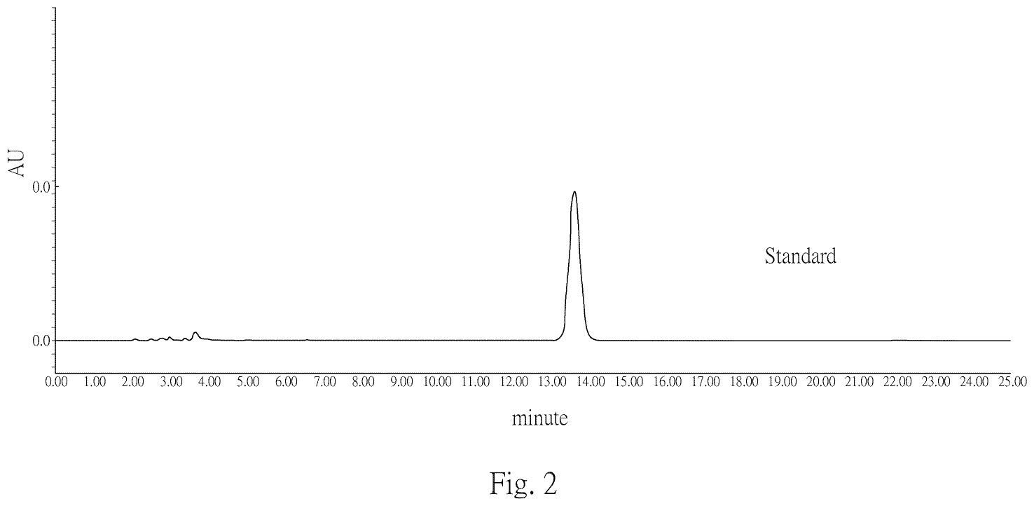

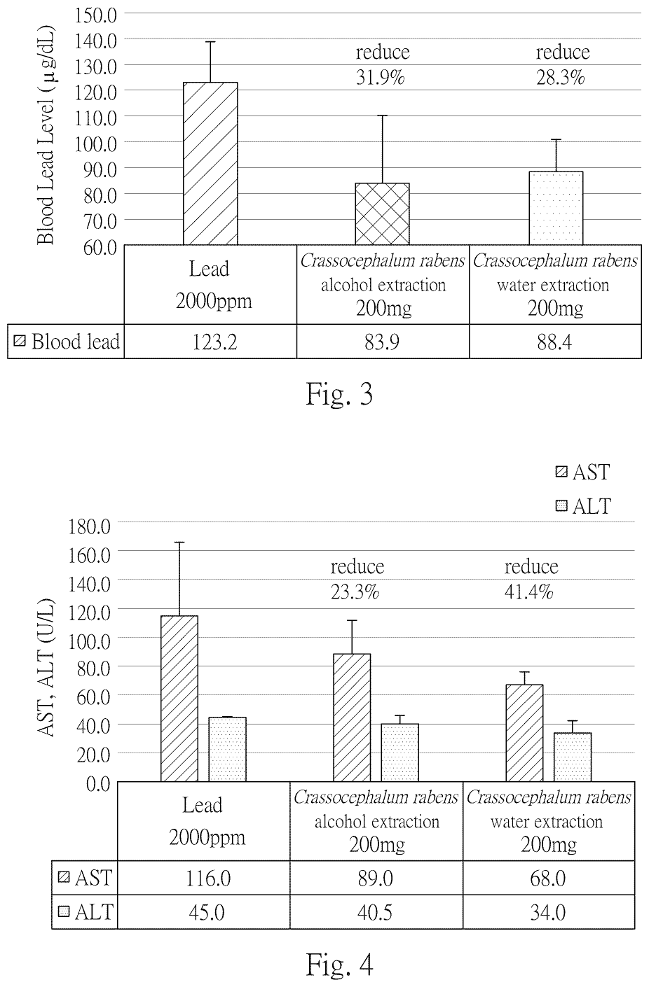

Six-week-old SD rats were fed daily with 1 mL of 2000 ppm lead acetate aqueous solution. The two groups of rats other than the blank control group were each given, on a daily basis in addition to the lead acetate aqueous solution, a Crassocephalum rabens water extract sample or a Crassocephalum rabens alcohol extract sample in an amount equivalent to 200 mg/day for a human adult. After 14 days, blood was collected from the rats in order to analyze the blood lead concentrations and certain liver indices. The analysis results are shown in FIG. 3 and FIG. 4.

It can be known from the results in FIG. 3 and FIG. 4 that after drinking high-lead water for 14 days, the rats in the blank control group had a significantly high blood lead concentration, accompanied by a significantly high aspartate aminotransferase (AST) level and alanine aminotransferase (ALT) level. This indicates that continuous administration of lead ions to an individual will cause not only an accumulation of lead because lead cannot be metabolized in the individual′ body, but also damage to the individual's liver functions. By contrast, the administration of the Crassocephalum rabens water extract sample or the Crassocephalum rabens alcohol extract sample to rats exposed to a high-lead environment can effectively reduce the blood lead concentrations and prevent the liver function indices from lowering.

The results of this example prove that the Crassocephalum rabens extract disclosed herein does have the activity to remove the heavy metal (e.g., lead) in an individual's body. In other words, the administration of an effective amount of the Crassocephalum rabens extract disclosed herein or of a composition containing the Crassocephalum rabens extract can effectively treat or prevent lead poisoning and liver damage or liver diseases caused by exposure to an excessive amount of heavy metal.

1. A method for heavy metal detoxification by Crassocephalum rabens extract, comprising administering to a mammal a composition comprising a therapeutically effective amount of Crassocephalum rabens extract to lower a heavy metal concentration in the blood and improve the liver function, and thereby to treat and/or prevent heavy metal poisoning and the related disease thereof.

2. The method of claim 1, wherein the Crassocephalum rabens extract is a mixture rich in 1,2-di-O-linolenoyl-3-O-β-galactopyranosyl-sn-glycerol (dLGG).

3. The method of claim 1, wherein the mammal has a heavy metal poisoning caused by exposure to lead or to a substance containing lead ions.

4. The method of claim 1, wherein the composition is used to reduce an aspartate aminotransferase (AST) level and an alanine aminotransferase (ALT) level in blood.

5. The method of claim 1, wherein the Crassocephalum rabens extract is obtained by performing an extraction process on Crassocephalum rabens, and the extraction process uses water or a primary alcohol as an extraction solvent.

6. The method of claim 5, wherein the primary alcohol is ethanol having a concentration of at least 95%.

7. The method of claim 1, wherein the heavy metal is lead.

8. The method of claim 1, wherein the mammal is human.

9. The method of claim 8, wherein the therapeutically effective amount of Crassocephalum rabens extract is 100-250 mg/day.

10. The method of claim 9, wherein the therapeutically effective amount of Crassocephalum rabens extract is 200 mg/day.

11. The method of claim 1, wherein the heavy metal poisoning related disease is a liver disease.
